(19) United States
(12) Patent Application Publication
(10) Pub. No.: US 2006/0155240 A1
(43) Pub. Date: Jul. 13, 2006

(54) Pessary applicator providing low placement

(76) Inventors: Thomas Ward Osborn III, Cincinnati, OH (US); Diana Lynne Gann, Lebanon, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/298,092

(22) Filed: Dec. 8, 2005

Related U.S. Application Data
(63) Continuation-in-part of application No. 10/860,921, filed on Jun. 4, 2004.

(51) Int. Cl. A61F 13/20

(52) U.S. Cl. 604/15

(57) ABSTRACT

A pessary applicator for positioning a pessary inside a vaginal cavity. The pessary applicator includes an outer member, an inner member which is slidable within the outer member, and a pessary which is housed within the pessary applicator. The inner member has an effective length of less than about 58 mm.

### CROSS REFERENCE TO RELATED APPLICATIONS

This application is a continuation-in-part of U.S. application Ser. No. 10/860,921, filed Jun. 4, 2004, pending.

### FIELD OF INVENTION

This invention relates to a pessary applicator, particularly, to a pessary applicator providing low placement of a pessary within the vaginal cavity.

### BACKGROUND OF THE INVENTION

Urinary incontinence is a widespread problem among females. It is estimated that up to 50% of women occasionally leak urine involuntarily, and that approximately 25% of all women will seek medical advice at some point in order to deal with the problem. The two primary types of incontinence are stress incontinence and urge incontinence which have different origin and can occur as separate or combined conditions. Treating one condition does not necessarily mean the other condition has been treated. Incontinence is more prevalent among women who have given birth to one or more children. There are two primary demographic factors that are associated with female urinary incontinence: Child birth and aging.

Stress incontinence, the most common type of urinary incontinence, refers to the involuntary loss of urine resulting from abdominal pressure rise, occurring during exercise, coughing, sneezing, laughing, etc. When stress incontinence occurs, it is usually the result of the abnormal descent of the urethra and bladder neck below the level of the pelvic floor. While many different factors may contribute to the development of stress incontinence, stress incontinence is both aggravating and unpleasant for women, and it can also be embarrassing. Many women wear sanitary pads or diapers in order to deal with incontinence, though this is not a real solution to the problem. Diapers and pads are inconvenient, have side effects of skin irritation and odor limiting her social involvement and reducing her self esteem. Some cases of incontinence are treated surgically but have the normal risks associated with surgery, such as infection, and results are often not permanent.

One modality for non-surgical treatment used to reduce or prevent urinary incontinence in women includes devices which are located in the opening of the urethra and block the same. Shortcomings of these types of devices include the fact that they must be removed in order to empty the bladder and replaced with a new device which proves inconvenient and costly, they are susceptible to causing infection, and they often cause irritation to body tissue.

Another modality of non-surgical treatment involves the use of non-absorbent devices inserted into the vagina, either by a medical practitioner or by the woman herself. These devices are called pessaries. Pessaries are commonly used for the management of uterine prolapse and to provide vaginal support of the bladder or rectum. Many pessaries are designed to apply pressure against the bladder neck so as to inhibit or completely block the flow of urine through the urethra. A variety of such devices are known in the art for example, U.S. Pat. No. 5,618,256 to Reimer, entitled, “Device for Arrangement in the Vagina for Prevention of Involuntary Urination with Females and an Applicator for use in Insertion of the Device;” U.S. Pat. No. 5,785,640 to Kresch, entitled “Method for Treating Female Incontinence;” U.S. Pat. No. 4,920,986 to Biswas, entitled, “Urinary Incontinence Device;” U.S. Pat. No. 5,417,226 to I Juma, entitled, “Female Anti-Incontinence Device;” U.S. Pat. No. 5,386,836 to Biswas, entitled, “Urinary Incontinence Device;” and U.S. Pat. No. 5,007,894 to Enhorning, entitled, “Female Incontinence Device.” A number of devices are constructed so as to completely block the urethra and thus need to be removed or collapsed in order to allow the woman to urinate.

There are several important unaddressed needs associated with pessaries. These include reproducible and proper positioning of the pessary within the vagina by the consumer which is essential for the device to effectively and reliably relieve the symptoms associated with urinary incontinence. Proper placement is also important for comfort.

Recently there have been several pessary designs that have utilized conventional pessary type applicators to place pessaries within the vagina (WO2004/103213 and U.S. Pat. No. 6,090,099). This approach has the significant disadvantage of not reliably controlling the insertion of the pessary into the vagina where it comfortable and effective. Current pessary applicators place the pessary above the cervix where a pessary is not effective because it neither supports the prolapsed uterus nor does it apply pressure against the urethra nor does it help to straighten any “kinks” in the urethra. Furthermore, if the pessary is placed too low, it can become exceedingly uncomfortable. This approach is applicable to pessaries that are obtained by prescription and those purchased directly by the consumer. The approach is equally applicable to disposable and non-disposable pessary devices.

Tampon applicators adapted to the insertion of vaginal pessary devices have been used for inserting a pessary into a vaginal cavity. In the field of pessary device applicators, “push” type pessary applicators are known. These applicators generally comprise, in a simple form, a pair of coaxial hollow cylinders or tubes. The larger outer tube serves as a vaginal insertion tube and houses a pessary. The smaller inner tube serves as a plunger which ejects the pessary from the outer tube.

It has been recognized that pessary applicators of the type described above have certain deficiencies which can be readily apparent to many users of such devices. In general, the position of the pessary within the vaginal cavity can directly impact the pessary's efficiency of reducing and/or eliminating urine leakage. For example, conventional “push” type applicators can place a pessary too high in the vaginal cavity forcing the device to the lateral side of the cervix. If the pessary is placed too high in the vagina and lodges to one side of the uterus, the reduction of urine leakage is not substantially improved because it cannot effectively support the urethra, uterus and bladder.

In addition, current pessary applicators are designed to “push” the pessary out of the outer tube substantially higher than the tip of the outer tube. This often causes the pessary to be deflected to one side of the vagina resulting in an off center position in the vaginal cavity.

Alternatively, pessary applicators can place a pessary too low in the vaginal cavity. When the pessary is placed too low in the vaginal cavity, the pessary can cause bodily discomfort to the wearer because of the pressure exerted from the vaginal sphincter muscles.

To solve the above problem of positioning the pessary, it would be beneficial to provide a pessary applicator which properly places the pessary at a desired position in the vaginal cavity.

### SUMMARY OF THE INVENTION

The present invention encompasses a pessary applicator for positioning a pessary inside a vaginal cavity. The applicator includes an outer member and an inner member slidable within the outer member. The inner member has an effective length of less than about 58 mm. The pessary is housed within the pessary applicator.

All documents cited are, in relevant part, incorporated herein by reference; the citation of any document is not to be construed as an admission that it is prior art with respect to the present invention.

### BRIEF DESCRIPTION OF THE DRAWINGS

FIG. 1 is a perspective view along a longitudinal axis of a pessary applicator of the present invention.

FIG. 2 is an exploded view of the pessary applicator along the longitudinal axis illustrative of the present invention before the assembly of the pessary applicator.

FIG. 3a is a perspective view of a pessary applicator of the present invention.

FIG. 3b is a cross-sectional view along line 3b-3b of the pessary applicator of FIG. 3a.

FIG. 4 is a perspective view of the pessary applicator of the present invention fully engaged.

FIG. 5 is a perspective view of an alternative embodiment of the pessary applicator of the present invention fully engaged.

FIG. 6a is a perspective view of an alternative embodiment of the pessary applicator of the present invention.

FIG. 6b is a perspective view of an alternative embodiment of the pessary applicator of the present invention fully engaged.

FIG. 6c is a perspective view of an alternative embodiment of the pessary applicator of the present invention fully engaged.

FIG. 7a is a perspective view of an alternative embodiment of the pessary applicator of the present invention.

FIG. 7b is a perspective view of an alternative embodiment of the pessary applicator of the present invention fully engaged.

FIG. 7c is a perspective view of an alternative embodiment of the pessary applicator of the present invention fully engaged.

### DETAILED DESCRIPTION OF THE INVENTION

Section A will provide terms which will assist the reader in best understanding the features of the invention but not to introduce limitations in the terms inconsistent with the context in which they are used in this specification. These definitions are not intended to be limiting. Section B will discuss the pessary applicator of the present invention. Section C will discuss the pessary applicator materials.

A. Terms

As used herein, the term “pessary” refers to any type of non absorbent or minimally absorbent vaginal insert for the purpose of reducing urine leakage. Such pessaries may be have any variety of shapes and sizes including cylinder, ovate, spherical, tubular, annual rings, “U” shaped, cup shaped, rings, cubes or donut shaped. They function by direct application of support, level force, expansion of the device by selection of material or by inflation of the device.

The term “fully engaged,” as used herein, occurs when the second end of the inner member is flush with the second end of the outer member or after 1250 grams of force has been applied to the end of the inner member.

The term “effective distance,” as used herein, is the distance that the trailing end of the pessary travels after the pessary pushing member contacts the pessary.

The term “effective length,” as used herein, is the end of the pessary to the second end of the outer member of the pessary applicator following full applicator engagement.

The term “stop,” as used herein, is meant when a member is used to prevent respective second ends of inner member and outer member from becoming flush with one another or when 1250 grams of force is applied to the inner member. One skilled in the art could imagine that the inner member or the outer member could have projections, nibs, ribs, nubs, etc. that can function to stop the inner member from being inserted into the outer member.

The term “exposed portion of the pessary,” as used herein, is meant the portion of the pessary that is revealed from the pessary applicator as measured from the leading end of the pessary to the insertion end of the outer member along the longitudinal axis when the applicator is fully engaged.

As used herein, the term “length of the outer member” refers to the length of the outer member measured along the longitudinal axis from the surface of the outer member which is closest to the body during insertion to the surface of the outer member which is furthest away from the body during insertion when the applicator is fully engaged.

As used herein, the terms “vaginal cavity” and “within the vagina” refer to the internal genitalia of the human female in the pudendal region of the body.

As used herein, the term “hymen ring” refers to the demarcation between the vaginal cavity and external genitalia and is identified by the position of the hymen or residual tissue of the hymen.

As used herein, the term “low placement” refers to a position of the pessary inside the vaginal cavity, wherein the trailing end of the pessary is positioned at least about 5 millimeters above the hymen ring and below the cervix.

The terms “expelled” and “expulsion,” as used herein, are meant the position of the pessary after the exposed portion of the pessary is fully or partially forced out of the pessary applicator. The diameter of the expelled portion of the pessary may be larger than the diameter of the portion of the pessary remaining in the applicator.

The term “joined” or “attached” encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element; i.e., one element is essentially part of the other element.

B. Pessary Applicator of the Present Invention

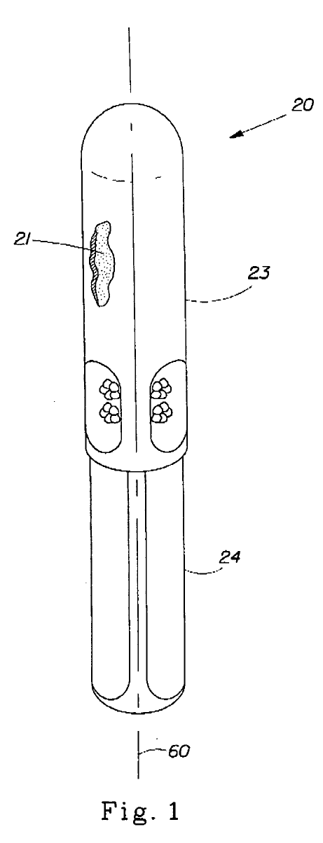

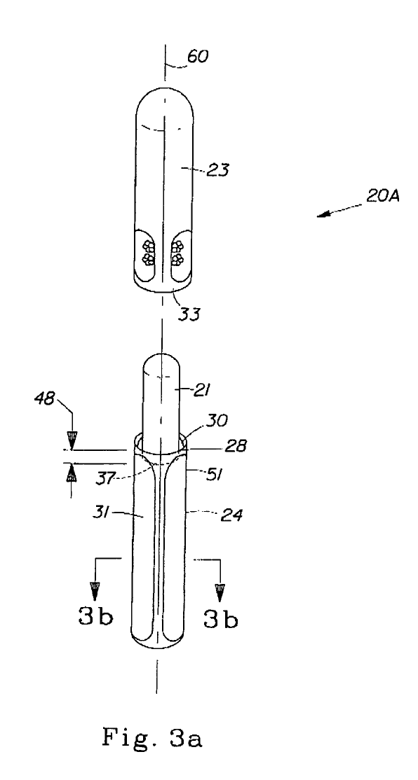

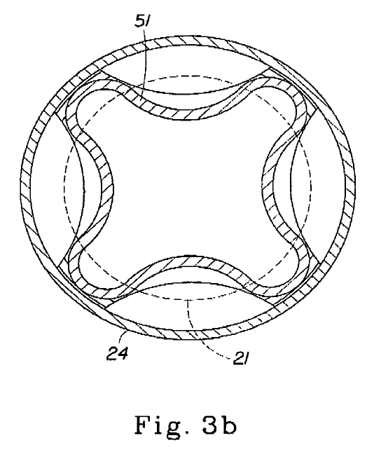

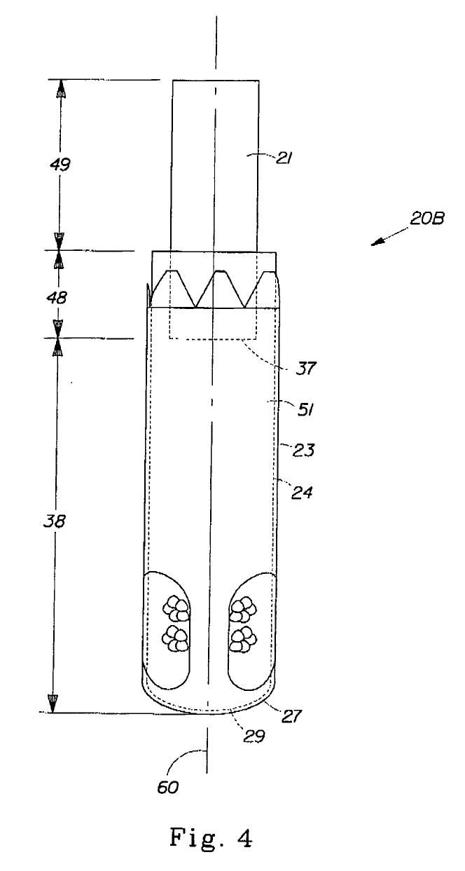

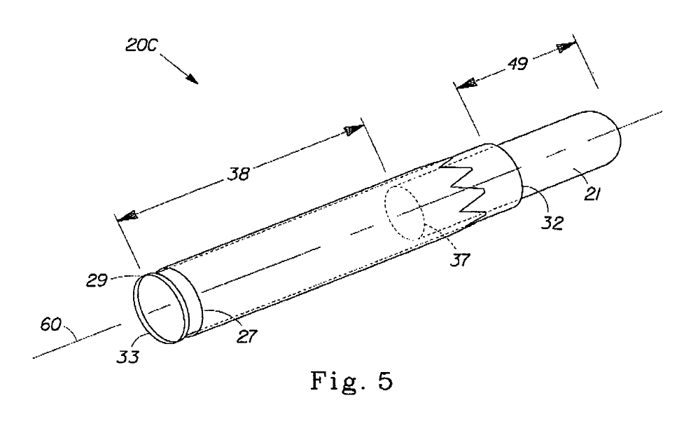

Referring to FIG. 1, a pessary applicator 20 is shown before expulsion of the exposed portion 49 (FIG. 4, FIG. 5) of pessary 21 along a longitudinal axis 60. The pessary applicator 20 is designed to position pessary 21 to achieve low placement in the vaginal cavity of a wearer. Generally, the pessary applicator 20 includes an outer member 23, an inner member 24, and a pessary pushing member 51 (see FIG. 3a).

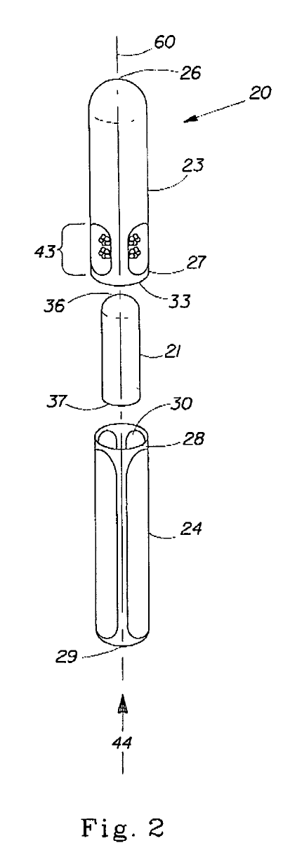

FIG. 2 is an exploded view of FIG. 1 of the pessary applicator 20 along the longitudinal axis 60. The inner member 24 having an effective length of less than 58 mm provides low placement of the pessary 21 within the vaginal cavity. In one non-limiting example, the effective length of the inner member is less than about 45 mm. In another non-limiting example, the effective length of the inner member is less than about 35 mm. In yet another non-limiting example, the effective length of the inner member is less than about 25 mm. Specifically, the effective length determines the placement depth of the trailing end 37 of the pessary 21 in the vaginal cavity. The trailing end 37 does not include any overwrap, secondary absorbent member, or withdrawal cord which extends beyond the pessary's main non-absorbent material. The outer member 23, the pessary 21, and the inner member 24 are shown. The outer member 23 can be used to insert the pessary 21 into the vaginal cavity. The outer member 23 comprises a hollow interior 33, an insertion end 26 dimensioned for insertion into the body cavity, and a second end 27 opposed to the insertion end 26. In addition, the outer member 23 can contain a grip region 43 located adjacent to the second end 27.

The pessary 21 has a leading end 36 and a trailing end 37. The leading end 36 of the pessary 21 is the end of the pessary 21 which is first inserted into the body. The trailing end 37 of the pessary 21 is opposed to the leading end 36 of the pessary 21. The pessary applicator 20 can be used with any type of pessary 21. For example, the pessary 21 could be a self-sustaining pessary or a deformable pessary.

The inner member 24 assists in the ejection of the exposed portion 49 (FIG. 4, FIG. 5) of the pessary 21 from the outer member 23 when a force 44 is applied along the longitudinal axis 60. The inner member 24 is dimensioned to slidably move within the outer member 23, with minimal clearance therebetween. The inner member 24 has a hollow interior 30. Alternatively, the inner member 24 can be solid or partially solid. The inner member 24 has a first end 28 and a second end 29 opposed to the first end 28.

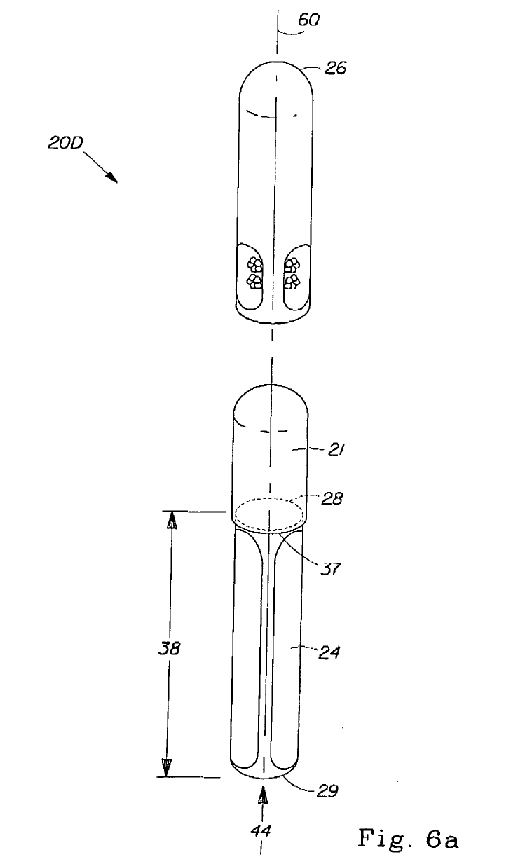

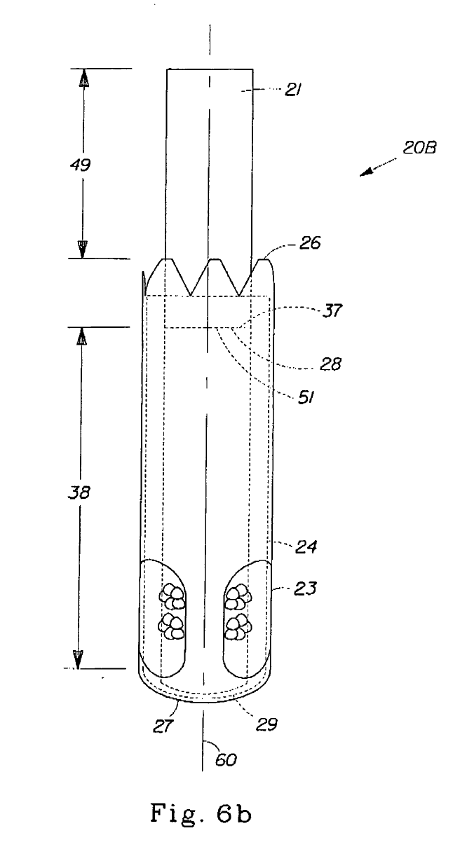

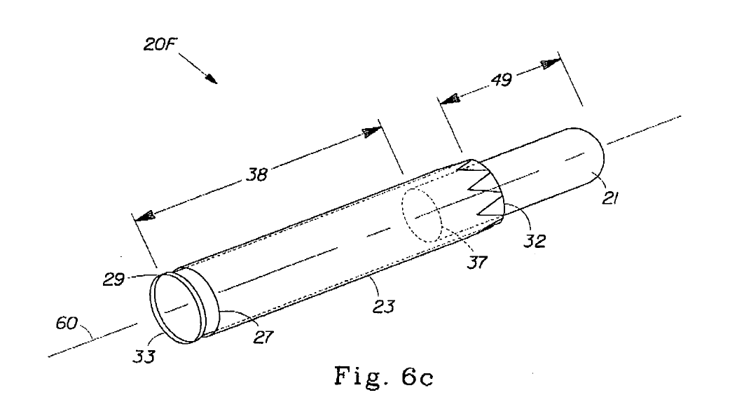

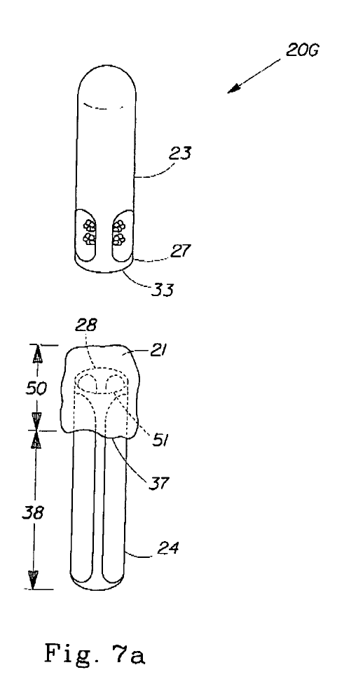

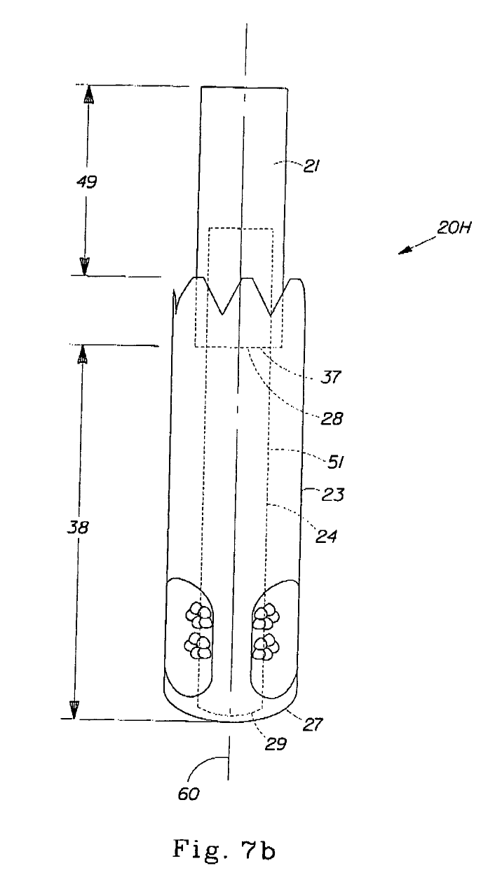

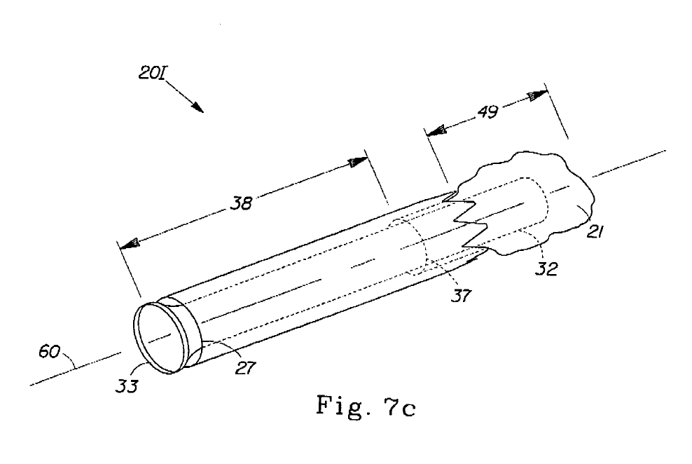

Below is a discussion of how the interaction of the pessary 21 and the inner member 24, 32 determines how the effective length 38 of the inner member 24 is measured. The pessary 21 could interact with the inner member in three ways. First, at least a portion of the pessary 21 could be embedded within the inner member 24 (See FIG. 3a). Second, the trailing end 37 of the pessary 21 could abut the first end 28 of the inner member 24 (See FIG. 6a). Third, the inner member 24 could be embedded in the pessary 21 (See FIG. 7a).

Pessary Embedded within Inner Member

FIG. 3a is a perspective view of the inner member 24 separated from the hollow interior portion 33 of the outer member 23. Also, as seen in FIG. 3a, the pessary 21 can be embedded in at least a portion of the inner member 24 during the expulsion and/or before the expulsion of the pessary 21 from the pessary applicator 20A. The length that the pessary 21 is embedded within the inner member 24 is the embedded pessary length 48.

Also, as shown in FIG. 3a, pessary applicator 20A could have a pessary pushing member 51. The pessary pushing member 51 is located in the hollow interior portion 30 of the inner member 24. The pessary pushing member 51 positions the pessary 21 and provides the necessary force to the pessary 21 such that during expulsion of the exposed portion 49 (FIG. 4, FIG. 5) of pessary 21, the pessary 21 will travel a pre-determined distance. The pessary pushing member 51 can be located in the inner member 24. The pessary pushing member 51 can be joined to the inner member 24. In one non-limiting example, as shown in FIG. 3a, the pessary pushing member 51 can be located adjacent to the first end 28 of the inner member 24 and the pessary pushing member 51 can be integral with the inner member 24. The pessary pushing member 51 in this limited embodiment can be created by four indented walls 31. The walls 31 can be indented along a portion of the length of the inner member 24. The portion of the inner member 24 which is indented creates a pessary pushing member 51 in which the trailing end 37 of the pessary 21 abuts the pessary pushing member 51 during expulsion of the exposed portion 49 (FIG. 4, FIG. 5) of pessary 21. In other words, an embedded portion 48 is contained within inner member 24 during expulsion of pessary 21.

FIG. 3b is a cross-section along lines 3b-3b of FIG. 3a. FIG. 3b shows pessary 21 adjacent to the pessary pushing member 51. The pessary pushing member 51 can be integral with the inner member 24. In this embodiment, pessary 21 is adjacent to the pessary pushing member 51.

FIG. 4 and FIG. 5 show alternative embodiments of the pessary applicator of the present invention when pessary 21 is embedded into inner member 24, 32 and when the pessary applicator is fully engaged resulting in the expulsion of the exposed portion 49 of the pessary 21. The pessary applicator of the present invention can be fully engaged when the second end 27 of outer member 23 becomes flush with the second end 29 of the inner member or after 1250 grams of force is applied to the inner member 32 along the longitudinal axis 60.

Referring to FIG. 4, when pessary 21 is embedded within the inner member 24 and when the inner member 24 becomes fully engaged by having second end 27 of the outer member 23 flush with the second end 29 of the inner member 24, the effective length 38 is the distance measured along the longitudinal axis 60 from the trailing end 37 of the pessary 21 to the second end 29 of the outer member 24. Alternatively, the effective length 38 can be calculated as the length of the inner member 24 minus the embedded pessary length 48. When pessary applicator 20B becomes fully engaged by having the second end 29 of inner member 24 becoming flush with second end 27 of the outer member 23, the exposed portion 49 of pessary 21 is expelled from pessary applicator 20B. In addition, when the second end 27 outer member 23 becomes flush with second end 29 of the inner member 24, the pessary 21 abuts the pessary pushing member 51 resulting in the exposed portion 49 expelling from pessary applicator 20B.

FIG. 5 shows pessary applicator 20C fully engaged after application of 1250 grams of force is applied to inner member 32 along the longitudinal axis 60. When the inner member 32 comes to a stop after applying 1250 grams of force to the inner member 32, the effective length 38 of the inner member 32 is the length as measured along the longitudinal axis 60 from the trailing end 37 of pessary 21 to the second end 27 of the outer member 23. In this embodiment, the inner member 32 comprises a hollow tube having a rim 33 which projects outwardly from the inner member 32. In addition, FIG. 5 shows an exposed portion 49 of pessary 21 after 1250 grams of force is applied to the inner member 32. In the embodiment of FIG. 5, after application of 1250 grams of force is applied to the second end 29 of inner member 32, the rim 33 of the inner member 32 prevents the inner member 32 from entering the outer member 23. In this embodiment, the rim 33 acts as a stop. A stop can be any feature that causes the inner member 32 to stop after 1250 grams of force is applied to the inner member 32.

b. Pessary Contacting Inner Member

FIG. 6a shows another alternative embodiment pessary applicator 20D. The first end 28 of the inner member 24 abuts the trailing end 37 of the pessary 21 during expulsion of the exposed portion 49 (see FIGS. 6b, 6c) of the pessary 21. The second end 29 is the portion of the inner member 24 in which the axial force 44 is applied along the longitudinal axis 60 to expel the exposed portion 49 (see FIG. 6b, 6c) of pessary 21 through the insertion end 26 of the outer member 23. The length of the inner member 24 is the effective length 38 in this embodiment.

FIG. 6b and FIG. 6c show alternative embodiments of the pessary applicator of the present invention when pessary 21 is not embedded into the inner member 24 and when the pessary applicator is fully engaged resulting in the expulsion of the exposed portion 49 of the pessary 21. The pessary applicator can be fully engaged when the second end 27 of the outer member 23 becomes flush with the second end 29 of the inner member 24 or after 1250 grams of force is applied to the inner member 32 along the longitudinal axis 60.

Referring to FIG. 6b, when the second end 27 of the outer member 23 becomes flush with the second end 29 of the inner member 24, the trailing end 37 of the pessary 21 abuts the first end 28 of the inner member 24 pushing the pessary 21 toward the insertion end 26 of the outer member 23 resulting in the exposed portion 49 expelling from pessary applicator 20B. In this embodiment, the first end 28 of the inner member 24 becomes the pessary pushing member 51. The length of the inner member 24 along the longitudinal axis 60 is the effective length 38 in this embodiment.

Referring to FIG. 6c, when the pessary 21 is not embedded into the inner member 32 and when the inner member 32 comes to a stop after applying 1250 grams of force to the inner member 32, the effective length 38 of the inner member 32 is the length measured along the longitudinal axis 60 from the trailing end 37 of the pessary 21 to the second end 27 of the outer member 23. In the embodiment of FIG. 6c, the point of stopping is the rim 33 which is the location which prevents the second end 29 of the inner member 32 from being inserted into outer member 23 after application of 1250 grams of force. The point of stopping may be different for different applicators. In this example, there can be a stop on the inner member 32 or the outer member 23. A stop can be any feature that causes the inner member 24 to stop after 1250 grams of force is applied. One skilled in the art could imagine that the inner member or the outer member 23 could have projections, nibs, ribs, nubs, etc. that can function to stop the inner member 24.

c. Inner Member Embedded within the Pessary

FIG. 7a is a perspective view of the inner member 24 separated from the hollow interior portion 33 of the outer member 23. Moreover, FIG. 7a shows inner member 24 embedded within pessary 21. The length that the inner member 24 is embedded within pessary 21 is the embedded inner member length 50. In this embodiment, the first end 28 becomes the pessary pushing member 51. The trailing end 37 of pessary 21 defines the location of the first end 28 of the inner member 24. The effective length 38 of the inner member 24 is the length from the trailing end 37 of pessary 21 to the second end 29 of the outer member 23. When the inner member 32 comes to a stop after applying 1250 grams of force to the inner member 24, the effective length of the inner member 32 is defined by the trailing end 37 of pessary 21 to the second end 27 of the outer member 23.

FIG. 7b and FIG. 7c show alternative embodiments of the pessary applicator of the present invention when inner member 24, 32 is embedded into the pessary 21 and when the pessary applicator 20H, 201 is fully engaged resulting in the expulsion of the exposed portion 49 of the pessary 21. The pessary applicator of the present invention can be fully engaged when the outer member 23 becomes flush with the inner member 24 or after 1250 grams of force is applied to the inner member 32 along the longitudinal axis 60. FIG. 7b shows pessary applicator 20H becoming fully engaged when the second end 29 of outer member 23 becomes flush with the second end 27 of inner member 24 resulting in the exposed portion 49 expelling from pessary applicator 20H. The effective length 38 of the inner member 24 is measured along the longitudinal axis 60 from the trailing end 37 of the pessary 21 which abuts the first end 28 of inner member 24 to the second end 29 of the inner member 24. In this embodiment, the first end 28 becomes the pessary pushing member 51.

FIG. 7c shows pessary applicator 201 fully engaged after application of 1250 grams of force is applied to inner member 32 along the longitudinal axis 60. The inner member 32 can comprise a hollow tube having a rim 33 which projects outwardly from the inner member 32. In addition, FIG. 7c shows an exposed portion 49 of pessary 21 after 1250 grams of force is applied to the inner member 32. In this embodiment, the effective length 38 of the inner member 32 is measured from the trailing end 37 of the pessary 21 which abuts the inner member 32 to the point when the inner member 32 stops being inserted into the outer member 24. In this embodiment, the rim 33 is the stop.

Below will provide more detail of each component of the pessary applicator of the present invention.

i. Outer Member

Referring to FIG. 2, typically, the outer member 23 can be used to handle or grip the pessary applicator during the insertion into the vaginal cavity. The outer member 23 is external to the inner member. The outer member 23 has a hollow interior 33, an insertion end 26, and a second end 27 opposed to the insertion end 26. The insertion end 26 is a portion of the outer member 23 in which the pessary applicator is inserted into the vaginal cavity and it is the end from which the pessary 21 is expelled. The second end 27 is opposed to the insertion end 26. The second end 27 can be the portion of the outer member 23 in which the user can handle or grip the pessary applicator. At least a portion of the hollow interior 33 of the outer member 23 engages with at least a portion of the inner member. In one non-limiting example, the inner member can be slideable within the outer member 23.

The manufacturer of the pessary applicator 20 can vary the size of the outer member 23. The size of the outer member 23 can be determined primarily by the dimensions of the pessary 21. Specifically, the diameter of the outer member 23 can be varied to accommodate different sized pessarys. Generally, the outer member 23 can include an inner diameter from about 6 millimeters to about 16 millimeters and a wall thickness of from about 0.4 millimeter to about 0.6 millimeter. Generally, the inner diameter of the outer member 23 should be suitably greater than the diameter of the pessary 21 to prevent the outer member 23 from interfering with the removal of the pessary 21 from the outer member 23. Moreover, the inner diameter of the outer member 23 can have varying diameters and shapes to conform to the profiled shape of the enclosed pessary 21.

Also, the manufacturer of the pessary applicator can vary the length of the outer member 23. Generally, the outer member 23 should be of a sufficient length to house at least a portion of the pessary 21 prior to the expulsion of the pessary 21 from the pessary applicator into the vaginal cavity. Generally, the length of the outer member 23 can be any length. In one non-limiting example, the length of the outer member 23 can be less than 25 mm. In another non-limiting example, the length of the outer member 23 can be less than 35 mm. In yet another non-limiting example, the length of the outer member 23 can be less than 45 mm. In yet another non-limiting example, the outer member 23 can be from about 50 millimeters to about 85 millimeters. In yet another non-limiting example, the length of the outer member 23 is preferably from about 50 mm to about 100 mm, more preferably from about 55 mm to about 85 mm.

In addition, the manufacturer of the pessary applicator can vary the shape of the outer member 23. It is further noted herein that the shape of the outer member 23 can vary as long as a portion of the inner member can be slideable within the outer member 23. Moreover, the outer member 23 is in no way limited by the shape that it can assume except that the shape should not hinder directional expulsion of the pessary 21. One skilled in the art can imagine that the outer member 23 might be cylindrical or curved like a banana or any other suitable shape as long as any such shape would work effectively to allow the exposed portion 49 (FIG. 4, FIG. 5) of pessary 21 to expel from the pessary applicator and comfortably insert the pessary 21 into the vaginal cavity. The outer member 23 can be of any suitable cross-sectional shape. For example, suitable cross-sectional shapes can include, but are not limited to, circular, oval, flattened circular, elliptical, and any combination thereof.

In addition, the second end 27 can also have many designs. For example, the second end 27 can be scalloped at the second end 27 and/or have a feathered thickness edge.

Furthermore, the outer member 23 can contain the grip region 43 located adjacent to the second end 27 as noted above. The grip region 43 can provide for secure handling of the outer member 23. The perimeter of the grip region 43 can take essentially any desired shape, including oval, circular, and various other geometric forms. The gripping region 43 can be defined by outward projections or raised surfaces, created by impressing or compressing the surfaces. As seen in FIG. 2, the grip region 43 can be substantially circularly shaped, but it can also take on more angular formations such as squared. The surface of the grip region 43 can be any kind of surface known in the art. This surface can provide a desired frictional resistance for the fingers during the insertion of the pessary applicator 20 into the body. Moreover, this surface area can have, for example, as seen in FIG. 1, a flower pattern created via surface texturing, impressions, and/or indentations.

Furthermore, it is preferable that the grip region 43 be of sufficient dimension to substantially provide a comfortable grip for the user. As used in the specification, the term “user's grip” means any way of holding the pessary applicator in a hand, e.g., between a thumb and a finger. The grip region 43 need not necessarily extend completely around the perimeter of the outer member 23. For example, a space can be provided for a decorative marking or a Trademark character.

ii. Inner Member

Generally, the inner member 24 has a hollow interior 30, a first end 28, and a second end 29 opposed to the first end 28. The second end 29 is opposed to the first end 28. Moreover, the second end 29 is the portion of the inner member 24 in which the axial force 44 is applied to expel the pessary 21 from the outer member 23.

The inner member can have many different sizes and shapes. One skilled in the art can imagine many shapes of the inner member 24, for example, cylindrical or curved like a banana or any other suitable shape possible. The inner member can be of any suitable cross-sectional shape including, but are not limited to, circular, oval, flattened circular, elliptical, and any combinations thereof. For example, referring to FIG. 3a, the inner member 24 can be a hollow member having four indented walls 31. FIG. 3a shows pessary 21 adjacent to the pessary pushing member 51. The inner member 24 is integral with the pessary pushing member 51.

In another example, as seen in FIG. 5, the inner member 32 can comprise a hollow tube having a rim 33 which projects outwardly from the inner member 32. In addition, FIG. 5 shows an exposed portion 49 of pessary 21 after 1250 grams of force is applied to the inner member 32.

In FIG. 3a, the inner member can be designed to store at least a portion of the pessary 21 within its hollow interior 30 either during the expulsion and/or before the expulsion of the pessary 21 from the insertion end 26 of the outer member 23. Notably, the inner member can be shaped such that at least a portion of the pessary 21 is housed in or is partially contained by the inner member before the expulsion or during the expulsion of the pessary 21 from the pessary applicator 20.

The pessary pushing member 51 can be joined to the inner member 24 in any way known in the art. Specifically, the pessary pushing member 51 can be joined to the hollow interior 30 of the inner member by any known means in the art. Alternatively, the pessary pushing member 51 can be integral with the inner member.

The manufacturer of the pessary applicator of the present invention can vary the shape of the inner member as long as any such shape would work effectively to contain the embedded portion 48 of the pessary 21.

The size of the inner member can be determined primarily by the dimensions of pessary 21. Specifically, the diameter of the inner member can be varied to accommodate different sized pessarys. For example, larger pessarys can have larger diameters resulting in the inner diameter of the inner member having a larger diameter to house the larger pessary. In addition, the inner diameter of the inner member can have varying diameters and shapes to conform to the profiled shape of the enclosed pessary 21.

The length of the inner member can also vary. Generally, the inner member can be of a sufficient length to house a substantial portion of the pessary 21 prior to the insertion of the applicator into the body, properly aiding in the expulsion of the pessary 21 from the pessary applicator 20, and comfortably inserting into the vaginal cavity. For example, the length of the inner member 24 can be from about 43 millimeters to about 90 millimeters.

iii. Pessary Pushing Member

Referring to FIG. 3a, the pessary pushing member 51 positions the pessary 21 along the length of the inner member 24 so that during expulsion the exposed portion 49 of the pessary 21, the pessary 21 will travel a pre-determined distance.

The pessary pushing member 51 can be fixedly joined to the inner member in any way known in the art. The pessary pushing member 51 can also be monolithic with the inner member, for example, by molding the pessary pushing member 51 and the inner member as one piece. In one non-limiting example, the pessary pushing member 51 can be fixedly joined to the inner member by friction fitting that snaps parts together, gluing, and/or melting. In another non-limiting example, the pessary pushing member 51 can also be a continuation of the inner member by folding or compressing a portion of the inner member 24 and turning the inner member inwards upon itself to create a tube inside a tube.

FIG. 3b is a cross-section along lines 3b-3b of FIG. 3a. FIG. 3b shows pessary 21 adjacent to the pessary pushing member 51. The pessary pushing member 51 is integral with the inner member 24. In this embodiment, the pessary 21 is adjacent to the pessary pushing member 51.

Referring to FIG. 3a during the expulsion, at least a portion of the pessary 21 is adjacent to the pessary pushing member 51. In other words, when the pessary applicator 20A is fully engaged, the pessary pushing member 51 is in contact with the pessary 21. However, the pessary 21 may or may not be in contact with the pessary pushing member 51 before the expulsion of the exposed portion 49 (FIG. 4, FIG. 5) of pessary 21 from the pessary applicator 20.

It is further noted herein that the shape of the pessary pushing member 51 can vary as long as the pessary pushing member 51 aids in positioning the pessary 21 along the length of the outer member 23 to provide proper placement of the pessary 21 within the vaginal cavity. In one embodiment, the pessary pushing member 51 includes an elongated pessary positioning member of a generally tubular shape wherein the first end of the elongated pessary positioning member abuts the pessary 21 during expulsion. Alternatively, the pessary positioning member can be a rod wherein the first end of the rod abuts the pessary 21 during expulsion. In another embodiment, the pessary pushing member 51 can be slideable within the outer member 23. In another alternative embodiment, the pessary pushing member can also comprise projections extending inward from the hollow interior 33 of the inner member 24. The projections can be of any shape or size as long as the projections allow the pessary 21 to be in contact with the projections during the expulsion. The projections can be fixedly joined to the outer member 23 in any way known in the art.

It is further noted herein that the length of the pessary pushing member 51 can vary as long as the pessary pushing member 51 aids the pessary 21 to be located in a position within the inner member 24 that will provide low placement within the vaginal cavity. In one non-limiting example, the length of the pessary pushing member 51 can be from about 38.1 millimeters to about 57.15 millimeters.

In a preferred embodiment, as shown in FIG. 3a, the pessary pushing member 51 is monolithic with the inner member 24, for example, by molding the pessary pushing member 51. In this embodiment, the pessary positioning member is created in the form of indented walls 31 on a portion of the inner member 24. The portion which is indented creates a seat for the pessary 21 to rest.

C. Pessary Applicator Materials

Different pessary applicator parts can be constructed from different materials and processes. The pessary applicator or any part of the pessary applicator can be formed of a spirally wound, convolutedly wound, or longitudinally seamed hollow tube that is formed from paper, paperboard, cardboard, or any combinations thereof. The pessary applicator or any part of the pessary applicator can also be injection molded, extruded, or formed from flexible plastic, such as thermoformed from plastic sheet or folded or wound from plastic film.

The pessary applicator or any part of the pessary applicator can be constructed from a single ply of material or be formed from two or more plies that are bonded together to form a laminate. The use of two or more plies or layers is preferred for it enables the manufacturer to use certain materials in the various layers that can enhance the performance of the pessary applicator or any part of the pessary applicator. When two or more plies are utilized, all the plies can be spirally wound, convolutedly wound, or longitudinally seamed to form an elongated cylinder. The pessary applicator or any part of the pessary applicator can be constructed using a smooth thin ply of material on the outside or exterior surface that surrounds a coarser and possibly thicker ply. When the pessary applicator or any part of the pessary applicator contains at least three plies, the middle ply can be the thicker ply, and the interior and exterior plies can be smooth and/or slippery to facilitate the expulsion of the pessary and to facilitate the insertion of the pessary applicator or any part of the pessary applicator into a woman's vagina. By sandwiching a thick, coarser ply of material between two thin, smooth plies, a pessary applicator or any part of the pessary applicator can be provided which is very functional. The pessary applicator or any part of the pessary applicator should contain one to four plies, although more plies can be utilized if desired.

The plies forming the pessary applicator or any part of the pessary applicator can be held together by an adhesive, such as glue, heat, pressure, ultrasonic, or any combinations thereof. The adhesive can be either water-soluble or water-insoluble. A water-soluble adhesive is preferred for environmental reasons in that the pessary applicator or any part of the pessary applicator will quickly break apart when it is immersed in water. Such immersion will occur should the pessary applicator or any part of the pessary applicator be disposed of by flushing it down a toilet. Exposure of the pessary applicator or any part of the pessary applicator to a municipal's waste treatment plant wherein soaking in water, interaction with chemicals, and agitation all occur, will cause the pessary applicator or any part of the pessary applicator to break apart and evenly disperse in a relatively short period of time.

### EXAMPLES

The following is a listing of examples illustrating various embodiments of the present invention. It would be obvious to those skilled in the art that various other changes and modifications can be made without departing from the spirit and scope of the invention.

### Example 1

The pessary applicator of the present invention can comprise the outer member of the length of 45 millimeters, the inner member of the length of 70 millimeters, a pessary of the length of 44 millimeters, and 13 millimeters of the pessary embedded within the inner member.

### Example 2

The pessary applicator of the present invention can comprise the outer member of the length of 25 millimeters, the inner member of the length of 50 millimeters, a pessary of the length of 35 millimeters, and 17 millimeters of the pessary embedded within the inner member.

### Example 3

The pessary applicator of the present invention can comprise the outer member of the length of 35 millimeters, the inner member of the length of 55 millimeters, a pessary of the length of 40 millimeters, and 15 millimeters of the pessary embedded within the inner member.

### Example 4

The pessary applicator of the present invention can comprise the outer member of the length of 40 millimeters, the inner member of the length of 52 millimeters, a pessary of the length of 48 millimeters, and 12 millimeters of the pessary embedded within the inner member.

### Example 5

The pessary applicator of the present invention can comprise the outer member of the length of 25 millimeters, the inner member of the length of 70 millimeters, a pessary of the length of 48 millimeters, and 30 millimeters of the inner member embedded within the pessary.

### Example 6

The pessary applicator of the present invention can comprise the outer member of the length of 35 millimeters, the inner member of the length of 40 millimeters, a pessary of the length of 42 millimeters, and 18 millimeters of the inner member embedded within the pessary.

### Example 7

The pessary applicator of the present invention can comprise the outer member of the length of 45 millimeters, the inner member of the length of 73 millimeters, a pessary of the length of 46 millimeters, a pessary positioning member of the length of 20 millimeters, and 16 millimeters of the inner member embedded within the pessary.

### Example 8

The pessary applicator of the present invention can comprise the outer member of the length of 45 millimeters, the inner member of the length of 73 millimeters, a pessary of the length of 46 millimeters, a pessary positioning member of the length of 57 millimeters, and 16 millimeters of the inner member embedded within the pessary.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A pessary applicator for positioning a pessary inside a vaginal cavity, said applicator comprising:
a.) an outer member;
b.) an inner member slidable within said outer member, wherein said inner member has an effective length of less than about 58 mm; and
c.) at least a portion of said pessary being housed within said pessary applicator.

2. The pessary applicator according to claim 1 wherein said pessary is housed within said inner member.

3. The pessary applicator according to claim 1 wherein said pessary is housed within said outer member.

4. The pessary applicator according to claim 1 wherein said inner member has an effective length of less than about 56 mm.

5. The pessary applicator according to claim 1 wherein said inner member has an effective length of less than about 53 mm.

6. The pessary applicator according to claim 1 wherein said inner member has an effective length of less than about 45 mm.

7. The pessary applicator according to claim 1 wherein said inner member has an effective length of less than about 35 mm.

8. The pessary applicator according to claim 1 wherein said inner member has an effective length of less than about 25 mm.

9. The pessary applicator according to claim 1 wherein said outer member has a length of less than about 45 mm.

10. The pessary applicator according to claim 1 wherein said outer member has a length of less than 35 mm.

11. The pessary applicator according to claim 1 wherein said outer member has a length of less than 25 mm.
